(19) United States
(12) Patent Application Publication
(10) Pub. No.: US 2025/0295503 A1
(43) Pub. Date: Sep. 25, 2025

(54) IMPLANTABLE ASSEMBLY FOR REMOVING FLUID FROM THE ABDOMINAL CAVITY

(71) Applicant: Ernesto Andrade, Miami Beach, FL (US)

(72) Inventor: Ernesto Andrade, Miami Beach, FL (US)

(21) Appl. No.: 18/670,507

(22) Filed: May 21, 2024

Related U.S. Application Data
(63) Continuation-in-part of application No. 17/531,595, filed on Nov. 19, 2021, now abandoned.
(60) Provisional application No. 63/115,880, filed on Nov. 19, 2020.

(51) Int. Cl. A61F 2/48; A61B 17/00; A61F 2/04

(52) U.S. Cl. CPC A61F 2/484; A61B 2017/00283; A61F 2002/046

(57) ABSTRACT

An assembly implanted in the body of the user and operative to remove fluid from the user's abdominal cavity including a major chamber, operative as a pump, structured for manual disposition between a collapsed orientation and an expanded orientation and a conduit assembly interconnecting the major chamber in fluid communication with the abdominal cavity and a user's bladder. A valve assembly connected to the conduit assembly in flow regulating relation to fluid passing from the abdominal cavity to the bladder, through the major chamber. The manual disposition of the major chamber is at least partially defined by a force applied thereto externally of the user body. Disposition of the major assembly between the collapsed and expanded orientations results in fluid flow from the abdominal cavity to the major chamber and successively from the major chamber to the bladder, for expulsion by urination.

### CLAIM OF PRIORITY

The present application is based on and a claim of priority is made under 35 U.S.C. Section 119(e) to a provisional patent application that is currently pending in the U.S. Patent and Trademark Office, namely, that having Ser. No. 63/115,880 and a filing date of Nov. 19, 2020, as well as to another currently pending and prior filed Non-Provisional patent application, namely, that having Ser. No. 17/531,595 filed on Nov. 19, 2021, the contents of which are both incorporated herein by reference in their entireties.

### BACKGROUND OF THE INVENTION

### Field of the Invention

This invention is directed to an assembly implanted in the body of a user and including a manually activated pump or chamber operative to remove and transfer excessive fluid from the user's abdominal cavity to the user's bladder, for expulsion from the user body through urination.

### Description of the Related Art

Exaggerated fluid accumulation in the abdominal cavity is called ascites, which is common in individuals with cirrhosis and usually develops when the liver begins to fail. In general, the development of ascites indicates advanced liver disease. Cirrhosis of the liver is the most common cause of ascites, but other diseases such as heart failure, kidney failure and infection or cancer can also cause ascites. Other cancer types also produce ascites and is known as carcinogenic ascites. In addition, ascites is caused by a combination of elevated pressure in the veins that travel through the liver (portal hypertension) and the disease in liver function caused by scarring of the liver or cirrhosis.

The majority of patients who develop ascites observe abdominal distention and rapid weight gain. Some people develop edema in the ankles and difficulty breathing due to the accumulation of excessive fluid around the lungs. Other symptoms or complications may occur and include abdominal pain and discomfort and difficulty in breathing which may be the direct result of excessive fluid accumulating in the abdominal cavity. Other complications include a limitation in the ability to eat, walk and perform normal daily activities.

Also, infection may result in spontaneous bacterial peritonitis (SBP) and usually causes abdominal pain, tenderness, fever and/or nausea. If not diagnosed or treated as soon as possible, the patient may develop renal failure, severe infection in the bloodstream as well as mental confusion.

In addition to the above hernia may be associated with ascites. More specifically elevated intra-abdominal pressure can lead to the development of umbilical (around the navel) and inguinal (groin) hernias that can cause abdominal discomfort and pain.

Accumulation of fluid in the chest is referred to as hepatic hydrothorax as the abdominal fluid fills the lung cavities. This disorder can result in difficulty breathing during activities or when at rest.

One of the most important steps in treating ascites is to strictly reduce salt intake. Very often nutritional methods of both water and salt intake fail resulting in the situation known as refectory ascites. In cases of this type peritoneal production find volumes of fluid in quantities over 5 L forcing medical personnel to do a surgical procedure, known as paracentesis. Such procedure typically involves a relatively long hospital stay and high cost associated with hospitalization, during which removal of liquid from the abdominal cavity is accomplished. As indicated, paracentesis is an invasive technique that, through an abdominal percutaneous puncture in order to evacuate fluid from the peritoneal cavity. Conventional period of hospital stay usually is in the range of two days. Further, this procedure must be performed by highly trained medical personnel in order to avoid infection, hemorrhage, hematoma, bacterial peritonitis, perforation of the intestine or bladder and/or vascular or neural injury.

Further disadvantages of invasive surgical procedures to remove excessive fluid within the abdominal cavity include the relatively minor complications of arterial hypertension, post-puncture abdominal pain, scrotal edema intestinal puncture without peritonitis and persistence of abdominal fluid flow through the needle insertion site. More serious or major complications include intraperitoneal hemorrhage, intestinal perforation and secondary peritonitis, abdominal wall abscess rupture of the puncture catheter in the peritoneal cavity or abdominal wall and abdominal wall hematoma.

Accordingly, there is a need in the medical arts and profession for a system, method and/or instrumentation for the removal of excessive peritoneal fluids without recourse to invasive surgical procedures. Such improved instrumentation and/or methodology could include an implantable biocompatible medical device or assembly intended to come in the direct contact with peritoneal fluids and be formed of a material which is resistant to such fluids therefore eliminating or restricting the requirement of replacement. Such a proposed system, method and biocompatible medical devices assembly would improve a patient's quality of life, minimizing medical complications secondary to ascites and greatly reduce the complications associated with paracentesis and decreasing costs and general discomfort associated with conventional medical procedures.

### SUMMARY OF THE INVENTION

The present invention is directed to an assembly, structured to be implanted in the body of a user or patient, and operative to remove fluid from a user's abdominal cavity. Therefore, as utilized and practiced, the assembly of the present invention may be utilized to remove excess peritoneal fluid from the abdominal cavity or peritoneum and as such may be accurately referred to as ascites pump assembly and/or a peritoneal pump assembly.

In more specific terms, the assembly of the present invention comprises a major chamber structured for manual disposition between a collapsed orientation and an expanded orientation. In addition, a conduit assembly serves to interconnect the major chamber in fluid communicating relation with the abdominal cavity and the user's bladder. Further, a valve assembly is connected to the conduit assembly in flow regulating relation to fluid passing from the abdominal cavity to the bladder, through the major chamber and concurrent to the aforementioned manual disposition between the collapsed orientation and the expanded orientation. As such, the manual disposition of the major chamber is at least partially defined by a manual force applied to the major chamber from an exterior of the user's body.

The major chamber is operatively positioned within the user body in accessible relation to an exterior of the user thereby serving to at least partially define the manual disposition of the major chamber by a force applied externally of the user, to the user skin or body part which is directly aligned with the internally implanted major chamber. The major chamber comprises an at least partially hollow interior and a flexible construction structured to be normally biased in the aforementioned expanded orientation concurrently to a removal or lessening of the external force applied to the major chamber to manually dispose it in the collapsed position. Therefore, the major chamber is operative to generate a negative pressure or vacuum on the interior thereof, concurrent to disposition of the major chamber from the collapsed orientation to the normally biased, expanded orientation.

The aforementioned valve assembly is disposed and structured to direct fluid flow from the abdominal cavity to and within the interior of the major chamber concurrent to generation of the negative pressure created on the interior major chamber as it moves from the collapsed orientation to the expanded orientation. The valve assembly comprises at least a first check valve disposed and structured to facilitate unidirectional fluid flow from the abdominal cavity to the major chamber, concurrent to the generation of the negative pressure on the interior of the major chamber. As indicated, the major chamber is operative to generate a positive pressure on the interior thereof concurrent to disposition of the major chamber from the expanded orientation into the collapsed orientation, when an external force is applied to the major chamber, as explained in greater detail hereinafter. In cooperation therewith, the valve assembly also comprises at least a second check valve disposed and structured to establish unidirectional fluid flow from the major chamber to the bladder, concurrent to the generation of the positive pressure on the interior of the major chamber, as it is being forced into the collapsed orientation.

The structural features of the aforementioned conduit assembly are such as to facilitate fluid flow from the peritoneum or abdominal cavity, through the interior of the major chamber and subsequently and successively to the bladder, wherein the fluid delivered to the bladder may be removed from the body through urination. Accordingly, the conduit assembly comprises at least a first conduit and at least a second conduit respectively defining a first path of fluid flow from the abdominal cavity to the major chamber and a second path of fluid flow from the major chamber to the bladder. The first and second conduits each include a free end respectively disposed in fluid communication with the interior of the abdominal cavity and the interior of the bladder. As such, each of the free ends are configured to restrict damaging engagement respectively with interior portions of the abdominal cavity, such as the abdominal mucosa, and the bladder, such as the peritoneal mucosa.

Accordingly, the cooperative structuring between the valve assembly and the conduit assembly comprises the one-way first check valve disposed along the first path of fluid flow and structured to establish unidirectional fluid flow from the abdominal cavity to the major chamber. Thereafter, the second one-way check valve is disposed along the second path of fluid flow and is structured to establish unidirectional fluid flow from the major chamber to the bladder, successively and subsequently to the interior of the major chamber being filled with the excess fluid collected from the interior of the abdominal cavity, through the first path of fluid flow defined by the at least first conduit.

Therefore, and as should be apparent, the collection and direction of fluid from the abdominal cavity along the first path of fluid flow defined by a first conduit and subsequently and successively from the major chamber to the bladder along the second path of fluid flow defined by the second conduit is a result of the major chamber being a manually disposed, by a force exerted and/or removed from an exterior of the user's body, between the collapsed and expanded orientations.

Yet additional structural operative details include the major chamber comprising a substantially reinforced or specifically structured peripheral portion which restricts the collapse of the peripheral portion as the major chamber is being manually disposed or forced from the expanded orientation into the collapsed orientation. In cooperation therewith, the collapsed orientation comprises a substantially central portion of the major chamber, surrounded by the peripheral portion of the major chamber, being disposed inwardly into a compressed position. Concurrently to the central portion of the major chamber being disposed into the substantially compressed position, the peripheral portion is disposed and/or will remain in the aforementioned, substantially non-compressed position, due to the reinforced structuring as set forth above. Such structuring and operative features facilitates fluid flow into the interior the major chamber and outwardly therefrom as the fluid collected from the abdominal cavity passes therefrom, through the interior the major chamber and into the bladder for disposal by urination.

In addition to the above, the assembly of the present invention may also include a minor chamber implanted within the user's body in an exteriorly accessible, fluid receiving position similar to the operative position of the major chamber. More specifically, a syringe or other injection instrument may pass from an external location through the skin of the user into the interior of the minor chamber for the delivery of medication, saline solution or other injected fluid, so as to be at least initially and temporarily stored within the interior of the minor chamber. In cooperation therewith, the aforementioned valve assembly and conduit assembly are structured to selectively deliver the fluid injected into the interior of the minor chamber to the interior of the abdominal cavity or peritoneal cavity. In one or more in additional embodiments, once the injected fluid is delivered into the interior of the abdominal or peritoneal cavity it may then pass along with the peritoneal fluid to the major chamber through the first conduit and/or first fluid flow path and therefrom into the bladder, through the second conduit or second fluid flow path, as the major chamber is manually disposed between the expanded and collapsed orientations.

In order to accommodate the injected fluid within the minor chamber being delivered to the interior of the abdominal cavity the valve assembly may include a supplementary valve which is structured to establish a unidirectional fluid flow from the interior of the minor chamber through the conduit assembly, independent of concurrent fluid flow from the abdominal cavity to the major chamber and/or from the major chamber to the bladder as explained herein.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a perspective view of the assembly of the present invention.

FIG. 2 is a detailed view of one component of the assembly of the embodiment of FIG. 1.

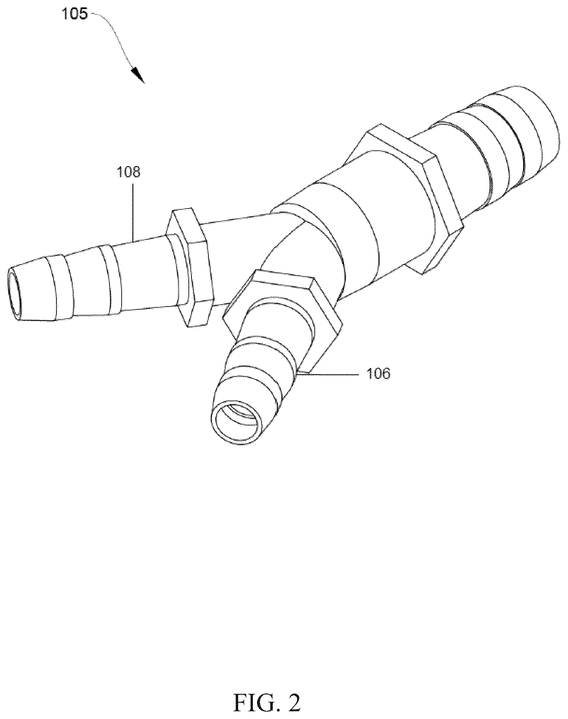

FIG. 3 is a detailed view of one component of a conduit assembly associated with the embodiment of FIG. 1.

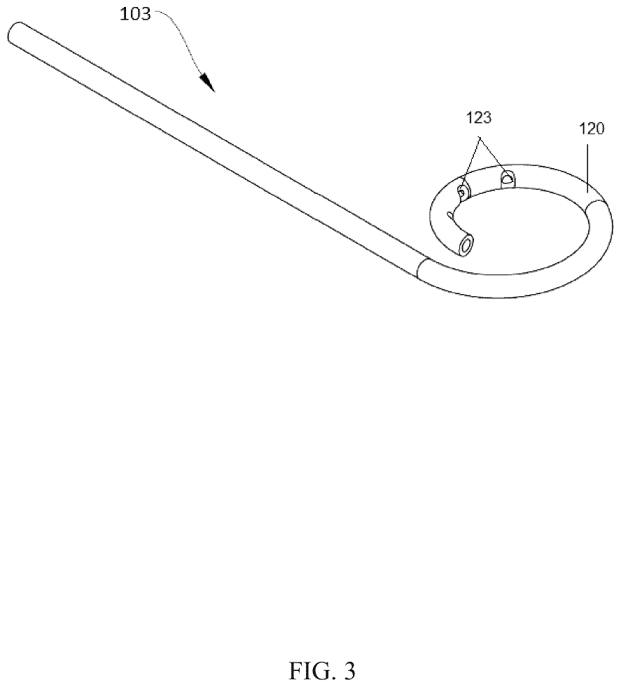

FIG. 4 is a detailed view of one other component of the conduit assembly associated with the embodiment of FIG. 1.

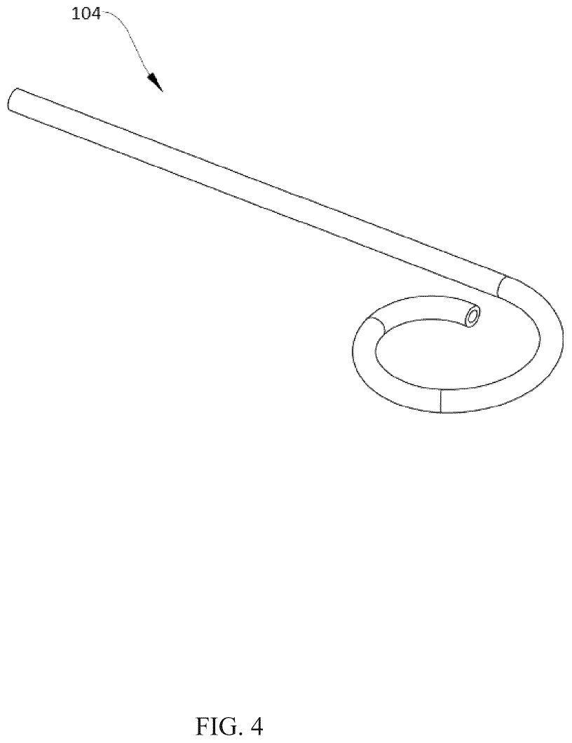

FIG. 5 is a detail, perspective view of a major chamber structure of the assembly of the embodiment of FIG. 1, in an expanded orientation.

FIG. 6 is a detail, perspective view of the major chamber structure of the embodiment of FIG. 5 in a collapsed orientation.

FIG. 7 is a detail, perspective view of a minor chamber of the assembly of the embodiment of FIG. 1.

FIG. 8 is a schematic detail view in partial cutaway of another embodiment of the major chamber structure which may be operatively associated with the assembly as represented FIG. 1.

FIG. 9 is a schematic representation of the operative positioning of at least a portion of the assembly as represented in FIG. 1 implanted in the body of a user.

FIG. 10 is a schematic representation of the operative features of the assembly as schematically represented in FIG. 9.

FIG. 11 is a schematic representation of another operative feature of the assembly as schematically represented in FIGS. 9 and 10.

FIG. 12 is a schematic representation of another operative feature of the assembly as schematically represented in FIGS. 9, 10 and 11.

FIG. 13 is a schematic representation of another embodiment of the assembly as also represented in FIG. 1.

FIG. 14 is a schematic representation of the embodiment of FIG. 13 in use.

Like reference numerals refer to like parts throughout the several views of the drawings.

### DETAILED DESCRIPTION OF THE PREFERRED EMBODIMENT

The invention now will be described more fully hereinafter with reference to the accompanying drawings in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

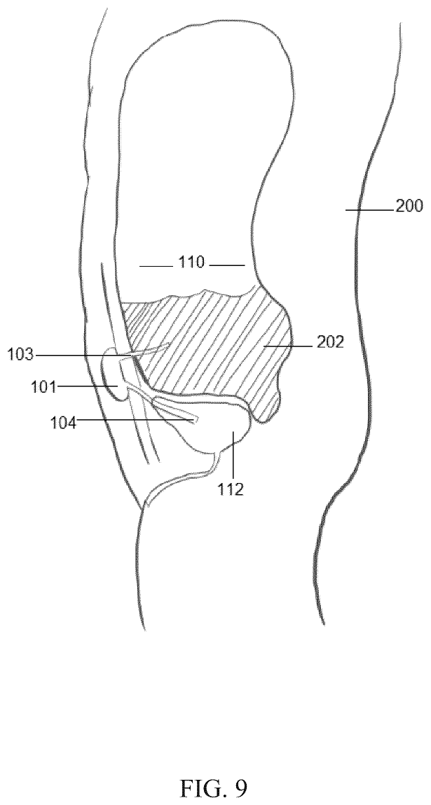

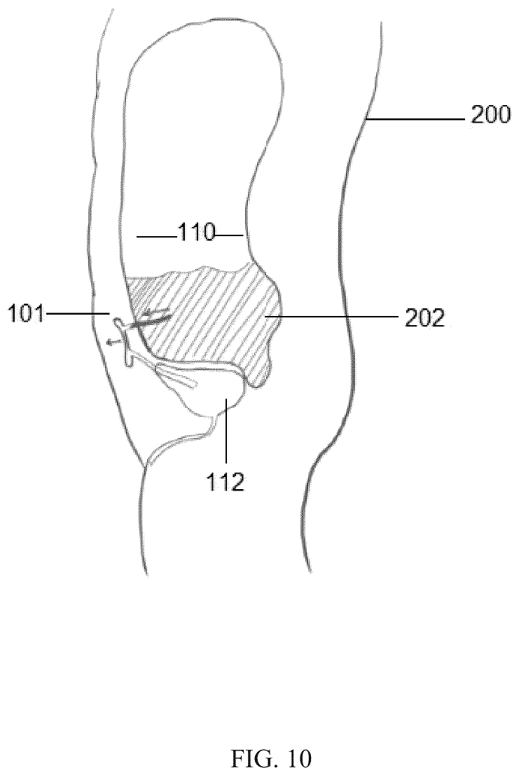

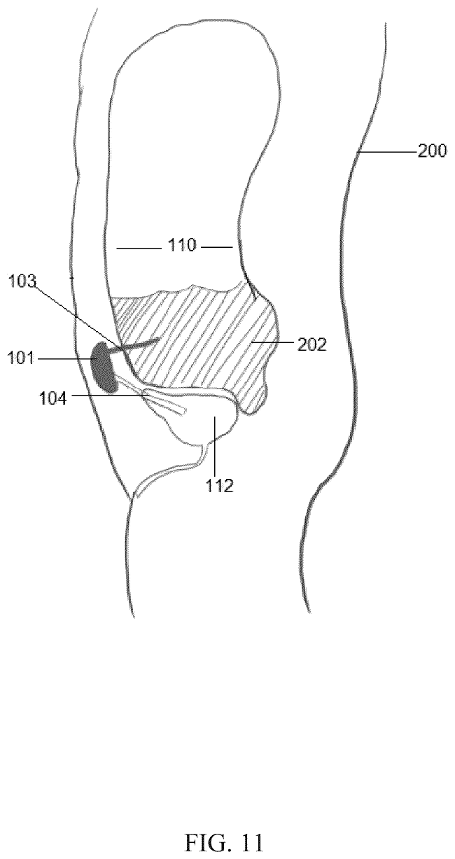

The present invention is directed to an assembly generally indicated as 100, structured to be implanted in the body of a user or patient 200 as schematically represented in FIGS. 9-14. Further, the assembly 100 is operative to remove fluid from a user's abdominal cavity. Therefore, as utilized and practiced, the assembly 100 of the present invention may be utilized to remove excess peritoneal fluid 202 from the abdominal cavity or peritoneum 110 and as such may be accurately referred to as ascites pump assembly and/or a peritoneal pump assembly, which delivers such collected excess fluid to the bladder 112 of the user (see FIGS. 9-12).

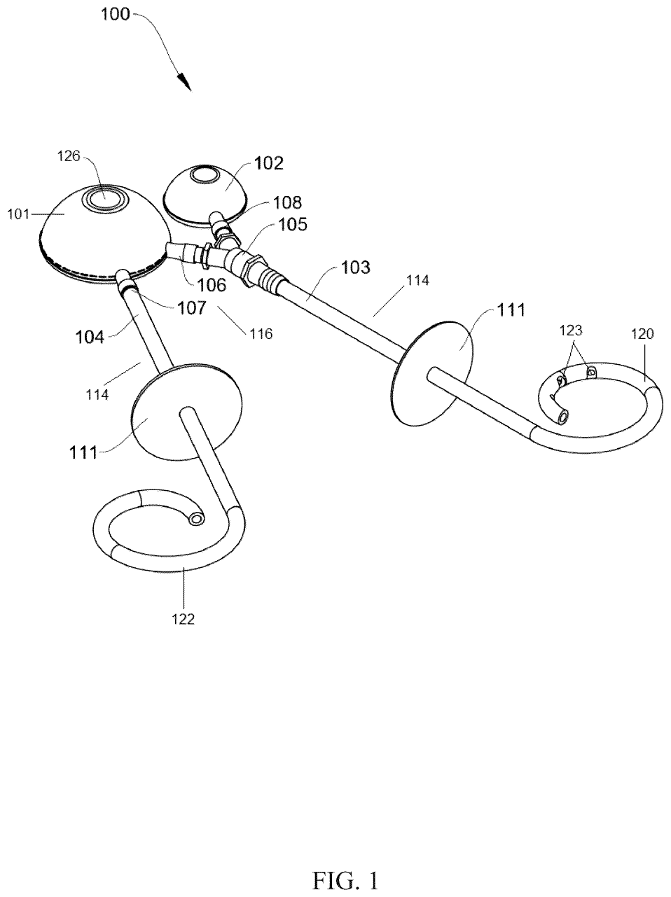

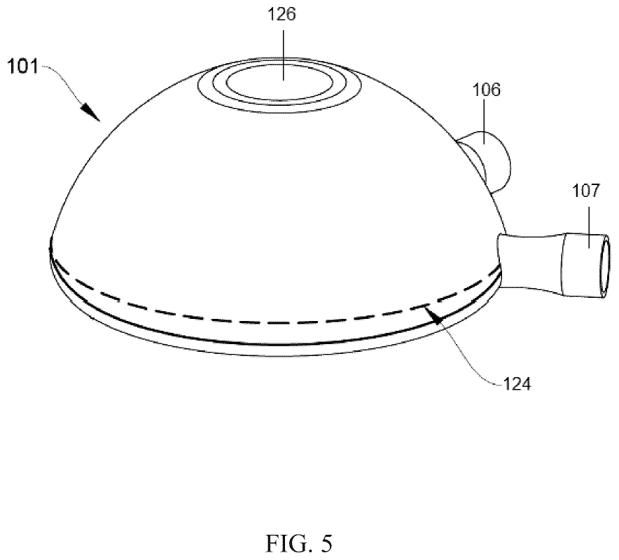

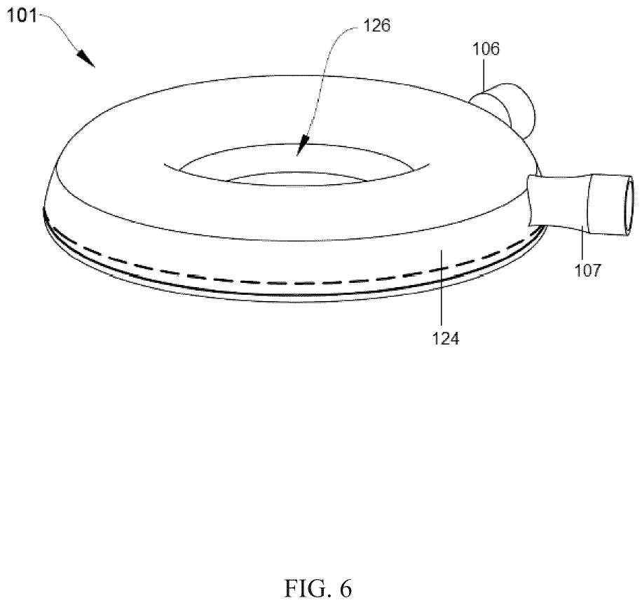

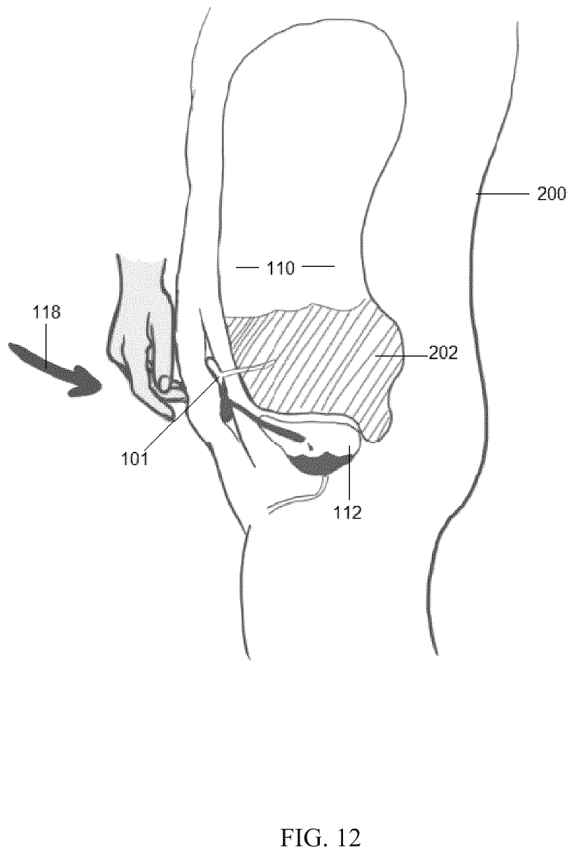

In more specific terms, the assembly 100 of the present invention comprises a major chamber 101 structured for manual disposition between a collapsed orientation, as represented in FIG. 6, and an expanded orientation as represented in FIGS. 1 and 5. In addition, a conduit assembly 114 serves to interconnect the major chamber 101 in fluid communicating relation with the abdominal cavity 110 and the user's bladder 112. Further, a valve assembly 116 is connected to the conduit assembly 114 in flow regulating relation to fluid passing from the abdominal cavity 110 to the bladder 112, through the major chamber 101, concurrent to the aforementioned manual disposition thereof between the collapsed orientation of FIG. 6 and the expanded orientation of FIGS. 1 and 5. As represented in FIG. 12, the manual disposition of the major chamber 101 is at least partially defined by a manual force 118, such as delivered by a hand of a user or other individual, being applied to the major chamber 101 from an exterior of the user's body. As also schematically represented the manual force 118 is delivered to the exterior skin or other body portion in aligned or corresponding position to the major chamber 101.

With further reference to FIG. 12, the major chamber 101 is operatively positioned within the user body in accessible relation to an exterior of the user. As such, the major chamber 101 is accessible to accomplish a manual disposition of the major chamber 101, by a force 118 applied externally of the user, to the user skin or body part which is directly aligned with the internally implanted major chamber 101. The major chamber 101 comprises an at least partially hollow interior and a flexible dome or hemispherical construction structured to be normally biased in the aforementioned expanded orientation of FIGS. 1 and 5 concurrently to a removal or lessening of the external force 118 applied to the major chamber 101 to manually dispose it in the collapsed position of FIG. 6. Therefore, the major chamber 101 is operative to generate a negative pressure or vacuum on the interior thereof, concurrent to the major chamber 101 moving from the collapsed orientation of FIG. 6 into the normally biased, expanded orientation of FIGS. 1 and 5.

The aforementioned valve assembly 116 is disposed and structured to direct fluid flow from the abdominal cavity 110 to and within the interior of the major chamber 101 concurrent to generation of the negative pressure created on the interior major chamber 101 as it moves from the collapsed orientation of FIG. 6 to the expanded orientation of FIG. 5. The valve assembly 116 comprises at least a first one-way check valve 106, disposed and structured to facilitate unidirectional fluid flow from the abdominal cavity 110 to the interior of major chamber, concurrent to the generation of the negative pressure on the interior of the major chamber 101. As indicated, the major chamber 101 is operative to generate a positive pressure on the interior thereof concurrent to a forced disposition of the major chamber 101 from the expanded orientation of FIG. 5 into the collapsed orientation of FIG. 6, when the external force 118 is applied to the major chamber 101, as schematically represented in FIG. 12 and explained in greater detail herein. In cooperation therewith, the valve assembly 116 also comprises at least a second one-way check valve 107 disposed and structured to establish unidirectional fluid flow from the major chamber 101 to the bladder 112, concurrent to the generation of the positive pressure on the interior of the major chamber 101, as it is being forced into the collapsed orientation of FIG. 6 by the manually delivered force 118 as schematically represented in FIG. 12.

The structural features of the aforementioned conduit assembly 114 are such as to facilitate fluid flow from the peritoneum or abdominal cavity 110, through the interior of the major chamber 101 and subsequently and successively from the interior of the major chamber 101 to the bladder 112, wherein the fluid delivered to the bladder 112 may be removed from the body through urination. Accordingly, the conduit assembly 114 comprises at least a first conduit 103 and at least a second conduit 104 respectively defining a first path of fluid flow from the abdominal cavity 110 to the major chamber 101 and a second path of fluid flow from the major chamber 101 to the bladder 112. The first and second conduits 103 and 104 each include a free end respectively indicated as 120 and 122. As utilized, the free ends 120 and 122 are respectively disposed within the interior of the abdominal cavity 110 and the interior of the bladder 112. Also, each of the free ends 120 and 122 are configured and structured to restrict damaging engagement respectively with interior portions of the abdominal cavity 110, such as the abdominal mucosa, and the bladder 112, such as the peritoneal mucosa. Such structural configuration of the free ends 120 and 122 may include a substantially curved or “pigtail” configuration, as represented. In addition, the free end 120 disposed within the abdominal cavity 110 may include a plurality of intake ports 123 disposed along a concave portion thereof so as not to engage or be otherwise exposed to the abdominal mucosa thereby at least partially preventing the clogging thereof as well as damage to the abdominal cavity.

Further, placement of the free end 120 on the interior of the abdominal cavity 110 is such as to be out of direct fluid communication with a normal or acceptable quantity of fluid therein. In contrast, when excessive fluid builds up in the abdominal cavity 110 it will “rise” or fill the peritoneum until the excess fluid is disposed in direct fluid communication with the free end 120 and the plurality of intake ports 123. As noted hereinafter in greater detail, absent an excess amount of fluid in the abdominal cavity 110, the major chamber 101, once depressed into a collapsed orientation, will not be filled with fluid, because the free end and intake ports are not in direct fluid communication with the fluid within the abdominal cavity 110. As a result, the major chamber 101 will remain in the collapsed orientation and will not automatically assume its normal expanded orientation, until filled.

In addition, at least one embodiment of the assembly 100 includes retaining structures 111 secured to the two conduits 103 and 104 between the corresponding ends 120 and 122 and the major chamber 101. The retaining structures 111 are disposed and structured to prevent or at least restrict inadvertent removal of the ends 120 and 122 from their intended communication with the abdominal cavity 110 and bladder 112.

Therefore, the cooperative structuring between the valve assembly 116 and the conduit assembly 114 comprises the first one-way check valve 106 disposed along the first path of fluid flow defined by the conduit 103 and structured to establish unidirectional fluid flow from the abdominal cavity 110 to the major chamber 101 along which the fluid 202 being collected travels. Thereafter and once the collected fluid 202 is within the interior of the major chamber 101, the second one-way check valve 107 is disposed along the second path of fluid flow, as defined by the second conduit 104, and is structured to establish unidirectional fluid flow from the major chamber 101 to the bladder 112. Such unidirectional fluid flow from the major chamber 101 to the bladder 112 occurs subsequently and successively after the interior of the major chamber 101 is filled with the collected excess fluid 202 from the interior of the abdominal cavity 110, through the first path of fluid flow defined by the at least first conduit 103.

As should be apparent, the collection and direction of fluid 202 from the abdominal cavity 110 along the first path of fluid flow defined by a first conduit 103 and subsequently and successively from the major chamber 101 to the bladder 112 along the second path of fluid flow defined by the second conduit 104 is a result of the major chamber being a manually disposed, by a force 118 (FIG. 12) exerted and/or removed from an exterior of the user's body 200, between the collapsed and expanded orientations as respectively represented in FIGS. 5 and 6.

Yet additional structural and operative details include the major chamber 101 comprising a substantially reinforced or specifically structured peripheral portion 124 which restricts or at least partially prevents the total collapse of the peripheral portion 124. As schematically represented in FIG. 12, the major chamber 101 is being manually disposed or forced from the expanded orientation of FIG. 5 into the collapsed orientation of FIG. 6. In cooperation therewith, the collapsed orientation of FIG. 6 comprises a substantially central portion 126 of the major chamber, 101 surrounded by the peripheral portion 124 being disposed inwardly into the interior of the major chamber 101 and into a compressed position as represented in FIG. 6. Concurrently to the central portion 126 of the major chamber 101 being disposed into the substantially compressed position of FIG. 6, the peripheral portion 124 is disposed and/or will remain in the aforementioned, substantially or at least partially non-compressed position, due to the reinforced structuring as set forth above. Such structuring and operative features may facilitate fluid flow into the interior the major chamber 101 and outwardly therefrom as the fluid 202 collected from the abdominal cavity 110, passes therefrom, through the interior the major chamber and into the bladder 112 for disposal by urination.

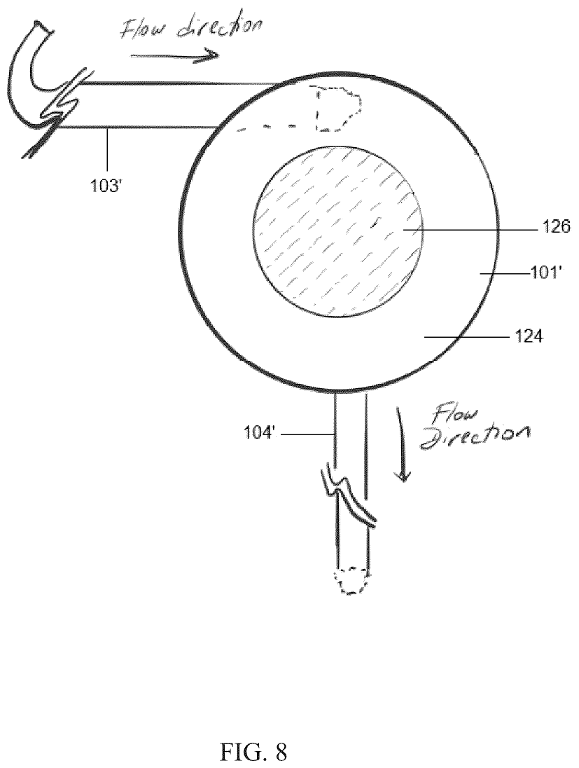

With initial reference to FIG. 8, at least one embodiment of the present invention includes the major chamber 101′ being connected to a first conduit 103′ in a substantially tangential relation or orientation as represented. Accordingly, the flow of fluid through the conduit 103′ to and along at least the non-compressed peripheral portion 124 of the major chamber 101′ facilitates delivery of fluid to the interior of the major chamber 101′. In cooperation therewith, the conduit 104′ may be connected to extend radially outward from the interior of the major chamber 101′, in fluid communication with the peripheral portion 124, thereby further facilitating outflow of fluid as it is delivered from the interior of the major chamber 101′ to the bladder 112.

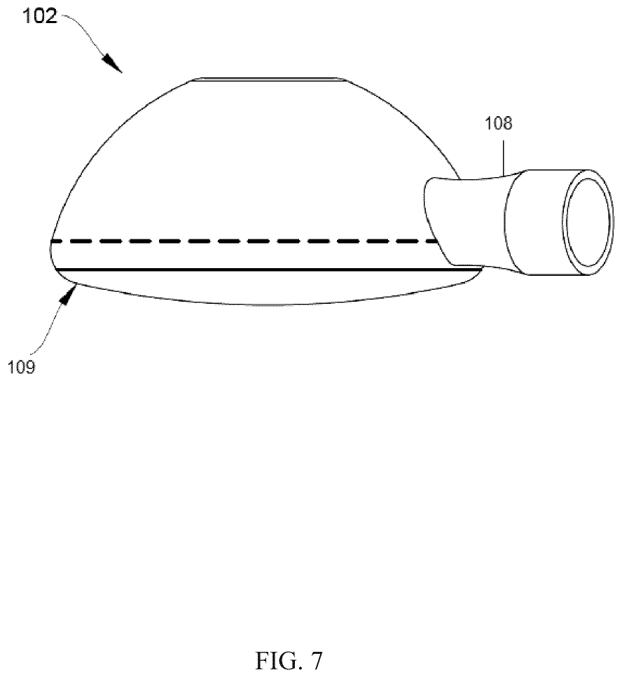

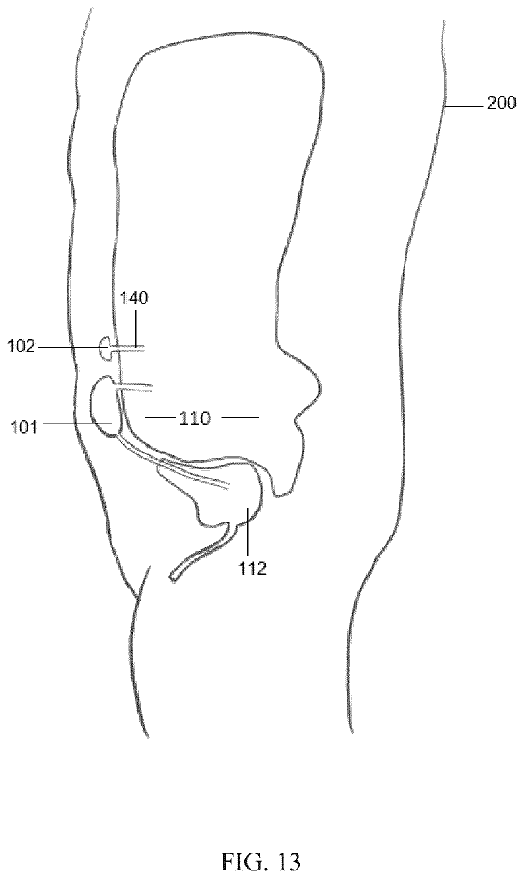

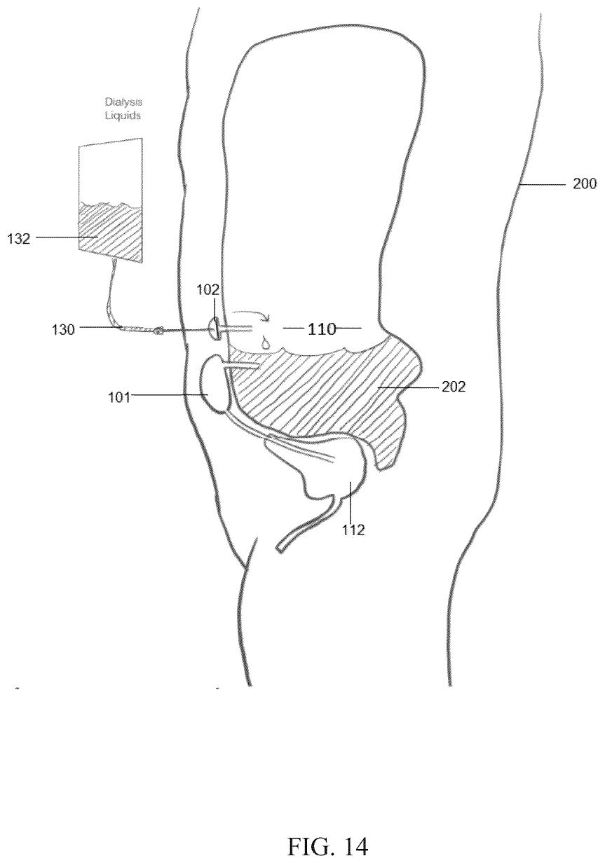

In addition to the above, the assembly 100 of the present invention may also include a minor chamber 102 (see FIG. 7) implanted within the user's body in an exteriorly accessible, fluid receiving position, schematically represented in FIGS. 13 and 14, similar to the operative position of the major chamber 101 as represented in FIGS. 9-12. More specifically, a syringe or other injection instrument 130 may pass from an external location through the skin of the user into the interior of the minor chamber 102 for the delivery of medication, saline solution or other injected fluid 132, so as to be at least initially and temporarily disposed within the interior of the minor chamber 102. As also represented in FIG. 7, the minor chamber 102 may have outer domed configuration made of a material which is “self-sealing” and preferably non-collapsible in contrast to the manner described herein relative to the major chamber 101. Such a non-collapsible feature facilitates penetration of the injection instrument repeatedly. Therefore, a needle from a syringe or other injection instrument 130 can repeatedly pass through the outer portion of the minor chamber 102 and upon removal, the penetration site will be automatically sealed.

As also represented in FIG. 7, the minor chamber includes a penetration resistant shield 109 disposed and structured to restrict passage of the injection instrument, from an interior of the minor chamber 102 therethrough, to an exterior thereof. In addition, the valve assembly includes a supplementary one-way check valve 108, which is oriented to establish unidirectional flow of injected fluid within the minor chamber 102 pass outwardly therefrom via split or bifurcated, Y-shaped connector 105. The Y-shaped connector 105 is disposed and configured to separately but concurrently interconnect the major chamber 101 and the minor chamber 102 to the first conduit 103 and first path of travel. The arrangement of the first one-way check valve 106 and the supplementary one-way check valve 108 is such as to force fluid flow from the interior of the minor chamber 102, upon the fluid 132 being forced therein by the injection instrument 130, into the Y-shaped connector 105 and conduit 103.

### Implantation

The assembly 100 is placed through a median infra umbilical incision. Skin and fat are dissected from the fascia of the rectus abdominis muscles. A midline incision is made in the facia between the rectus muscles. The peritoneum and bladder are identified and retracted. The major and minor chambers 101 and 102 respectively are located on the fascia of the patient's right rectus abdominal muscle, as is the valve assembly 116 including the check valves 106 and 108 operatively associated with the Y-shaped connector 105. With regard to the conduit assembly 114, first and second conduits 103 and 104 traverse the midline. An incision is made in the wall of the bladder 112, utilizing a #15 scalpel, and second conduit 104 with its flange 111 is inserted into the bladder. Next, a pintuck is made with prolene 4-0. The peritoneum 110 is retracted and perforated with a #15 scalpel blade, and first conduit 103 with its retaining structure 111 is inserted. A pintuck is made with Ethibon 3-0. The midline closes with Ethibon 3-0. The rest of the assembly 100 is left freely in the pocket created for this purpose, and the fat and skin are closed in layers, leaving an active drain to close the dead space.

### Operation and Use

The assembly 100 can be activated 48 hours after implantation. Subsequent to implantation, the assembly 100 is preferably for initial use is completely filled with sterile saline including the major chamber 101 and the minor chamber 102, as set forth hereinafter. When the patient, instructed by a physician, identifies an increase in volume in their abdominal cavity 110 due to general malaise, weight gain, and increased abdominal circumference, the assembly 100 can be used preferably in the physician's office or healthcare facility.

As represented in FIG. 12, the physician or user externally presses on the apex of the dome of the major chamber 101 until it completely collapses. This causes the first one-way check valve 107 to open. The check valve 107 is connected to the second conduit 104, defining the second path of fluid flow, emptying fluid within the major chamber 101 into the bladder 112. Therefore, pressing the dome of the major chamber 101 into a collapsed shape and/or orientation, as at least partially represented in FIG. 6, forces the saline solution and/or any liquid previously in the interior of the major chamber 101, through one-way check valve 107, and into the bladder 112 through second conduit 104. Once the pressure on the dome of the major chamber 101 is released, the major chamber 101 spontaneously returns to its normal expanded shape and/or orientation and causes the one-way check valve 107 to close, due to the negative pressure being developed on the interior of the main chamber 101. As indicated, the action of the dome of the major chamber 101 returning to its normal expanded shape and/or orientation causes a negative pressure (vacuum) to develop within the interior of the major chamber 101.

Substantially concurrently, the negative pressure developed within the main chamber 101 causes the one-way check valve 106, connected to the Y-shaped connector 105 to open and check valve 107 to close. The check valve 106 is connected to the first conduit 103, and under the influence of the negative pressure within the major chamber 101, the ascites fluid will pass from the peritoneum 110 into the intake ports 123. From there the ascites fluid passes through first conduit 103 and past the check valve 106 into the major chamber 101, resulting in the return of the dome and the major chamber 101 to assume its normal, expanded shape and/or orientation. Once the physician or user determines that the major chamber 101 has returned to its original expanded shape and/or orientation, external pressure again applied to the dome of the major chamber 101, forcing it into its collapsed shape and/or orientation. This pressure results in a closing of valve 106 and an opening of valve 107, resulting in displacement the peritoneal fluid within the major chamber 101 into the second conduit 104 and ultimately into the bladder 112, in the same way that the saline solution was previously displaced, as described above.

The physician will carry out this procedure as many times as necessary until discovering that the major chamber 101, once depressed into the collapsed shape and/or orientation, does not return to its original expanded shape and/or orientation and remains in the collapsed shape and/or orientation. This indicates that all and/or nearly all of the ascites fluid in the peritoneal cavity 110 has been drawn out and expelled in the bladder 112 as described. Notably, over time the body in due course will deposit more ascites fluid into the peritoneal cavity 110 which will slowly be drawn into the major chamber 101 under the remnant negative pressure. Typically, after a period of time in the range of about 2 hours, enough new ascites fluid will have been drawn into the major chamber 101 under the remnant negative pressure so that it returns to its normal expanded shape and/or orientation. At this time there is usually no need to drain that collected fluid into the bladder immediately, and instead it is acceptable and preferred to simply wait until the peritoneal cavity 110 re-fills beyond normal capacity and a regular drainage is desired, and/or at set normal intervals for draining the peritoneal cavity 110.

If desired, prior to the major chamber 101 re-filling with naturally produced ascites fluid, after the peritoneal cavity 110 has been substantially drained, and the major chamber 101 is in its collapsed shape and/or orientation, it may be desirable to disinfect and/or clean the major chamber 101 and the conduits. At that time, the physician can, using for example a 26-gauge needle and a syringe 130 with 50 ccs' of sterile saline solution, inject through the patient's skin and into the minor chamber 102 dome. Note that the minor chamber 102 has its base 109 preferably formed of a polycarbonate plate that prevents the needle from going through it and causing an intra-abdominal perforation. After placing the needle into the minor chamber 102, the saline solution or other disinfectant or cleaner can then be injected into the interior thereof. The positive force of the injected fluid into the interior of the minor chamber 102 will open the supplementary one-way check valve 108 and in the case where the major chamber 101 remains compressed, the negative pressure will draw the saline solution or other disinfectant or cleaner in, this cleaning the conduit and valves, and the interior of the major chamber 101. In some possibly preferred uses, the force from the syringe will cause at least some of the saline solution or other disinfectant or cleaner to pass into the first conduit 103 before the negative pressure created by the collapsed major chamber 101 draws the saline solution or other disinfectant or cleaner in, thereby resulting in cleaning of the conduit 103 as well. Once the saline solution or other disinfectant or cleaner fills and thereby returns the major chamber 101 to, or close to, its original expanded orientation, it is preferred that external pressure is once again applied to the major chamber 101 causing the saline solution or other disinfectant or cleaner along with any residue it has accumulated while cleaning, to pass out through valve 107 and into conduit 104 for passage to the bladder. This also returns the major chamber 101 to its collapsed shape and/or orientation so that it is ready to draw in more ascites fluid as it is generated by the body.

It is also understood that in some cases, especially if there is an amount of liquid within the major chamber 101, some of the injected fluid can pass into the abdominal cavity 110 to cause some level of cleaning and disinfecting therein. In that use case, the injected fluid will increase the volume of fluid within the abdominal cavity 110, thereby establishing direct fluid communication with the end 120. As before, because the major chamber 101 will be in the collapsed orientation the vacuum created therein as it assumes the expanded orientation will cause a delivery of the injected fluid (and any gathered or formed peritoneal fluid) to be delivered along the first conduit 103, back through check valve 106 and into the interior of the major chamber 101. The injected saline solution, mixed with peritoneal fluid from the abdominal cavity 110, will fill the major chamber 101 again, restoring the major chamber 101 to its original expanded orientation until it can be ejected into the bladder.

Either disinfecting process above can be repeated to leave inside the major chamber 101 a liquid as close to a sterile saline solution as practical. The procedure will be performed as many times as necessary so that, in the end, the major and minor chambers 101 and 102 respectively remain in position and are filled with saline solution. Here again, it is preferred that external pressure be applied once again applied to the major chamber 101 so that the saline solution or other disinfectant or cleaner along with any residue it has accumulated while cleaning passes out through valve 107 and into conduit 104 for passage to the bladder and returns the major chamber 101 to its collapsed shape and/or orientation so that it is ready to draw in more ascites fluid as it is generated by the body.

In another preferred use, a syringe can also be used to introduce medication such as antibiotics into the peritoneal cavity 110 and/or into the bladder 112. If they are to be introduced into the peritoneal cavity, which is less likely, it is preferred that the major chamber 101 be in its original expanded shape and/or orientation so that it does not quickly draw injected medication into the major chamber 101. Specifically, the syringe with the desired medication can be inserted into the minor chamber 102 as described, and the injected liquid passes through the conduit 103 into the peritoneal cavity 110. Alternatively, the antibiotic or other medication can be used during the process of re-filling the assembly 100 with saline for cleaning. Under that use, the antibiotic would move into the major chamber 101 and can be expelled into the bladder 112. The antibiotic would address infections propagating from the bladder 112. The antibiotic would be added to the same syringe 130 with the saline solution.

Finally, once the patient presents the same symptoms described, they will begin a new cycle of peritoneal fluid displacement to the bladder 112 to be eliminated from the body through urination. The assembly 100 establishes a method so that refractory large-volume ascites will not require paracentesis or show the symptoms and signs related to the intra-abdominal increase in peritoneal fluid.

Since many modifications, variations, and changes in detail can be made to the described and preferred embodiments of the invention, it is intended all matters in the foregoing description and shown in the accompanying drawings be interpreted as illustrative, exemplary, and non-limiting. For example, any use of the terms “preferably” or “preferred embodiment,” as well as other language akin thereto, is intended to refer to one particular embodiment, and solely one particular embodiment. As such, it may be appreciated other embodiments are possible, envisioned, and considered part of the invention disclosed herein. Thus, the scope of the invention should be determined by the appended claims and their legal equivalents.

What is claimed is:

1. An assembly implanted in a body of a user operative to remove fluid from the user's abdominal cavity, said assembly comprising:
a major chamber disposable between a collapsed orientation and an expanded orientation,
a conduit assembly interconnecting said major chamber in fluid communication with the abdominal cavity and a user's bladder,
said conduit assembly respectively defining a first path of fluid flow from the abdominal cavity to said major chamber and a second path of fluid flow from said major chamber to the bladder,
a valve assembly connected to said conduit assembly in flow regulating relation to fluid passing from the abdominal cavity to the bladder, via said major chamber,
a minor chamber disposed and structured to receive an injected fluid therein, via an injection instrument, and
said major chamber and said valve assembly cooperatively disposed and collectively operable to direct fluid flow from the abdominal cavity into said major chamber upon movement of said major chamber into said expanded orientation and from said major chamber into the bladder upon compression of said major chamber into said collapsed orientation, and to independently direct injected fluid from said minor chamber to said major chamber.

2. The assembly as recited in claim 1 wherein said valve assembly is disposed and structured to define independent fluid communication of said major chamber and said minor chamber with the abdominal cavity, via said first path of fluid flow.

3. The assembly as recited in claim 2 wherein said major chamber is disposable from said expanded orientation into said collapsed orientation concurrent to fluid flow from said major chamber into the bladder; said major chamber retained in said collapsed orientation concurrent to said first path of fluid flow disposed out of direct fluid communication with fluid within the abdominal cavity.

4. The assembly as recited in claim 3 wherein said major chamber is disposable from a retained collapsed orientation into said expanded orientation, upon the injected fluid from said minor chamber being disposed in the abdominal cavity.

5. The assembly as recited in claim 3 wherein said major chamber is disposable from a retained collapsed orientation into said expanded orientation, concurrent to being filled, at least in part, with the injected fluid from the abdominal cavity.

6. The assembly as recited in claim 3 wherein said major chamber is disposed into said expanded orientation from a retained collapsed orientation, concurrent to being filled with the injected fluid and peritoneum fluid.

7. The assembly as recited in claim 1 wherein said major chamber is operative to generate a negative pressure therein, concurrent to disposition thereof from said collapsed orientation to said expanded orientation; said major chamber operative to generate a positive pressure therein concurrent to disposition of the major chamber from said expanded orientation into said collapsed orientation so as to direct fluid out of said major chamber away from the abdominal cavity.

8. The assembly as recited in claim 7 wherein said valve assembly is disposed and structured to direct fluid flow in a single direction from the abdominal cavity into said major chamber, concurrent to said generation of said negative pressure.

9. The assembly as recited in claim 8 wherein said valve assembly comprises a first check valve disposed and structured to facilitate unidirectional fluid flow from the abdominal cavity to said major chamber, concurrent to said generation of said negative pressure.

10. The assembly as recited in claim 9 wherein said valve assembly comprises a second check valve disposed and structured to establish unidirectional fluid flow from said major chamber to the bladder, concurrent to said generation of said positive pressure.

11. The assembly as recited in claim 10 wherein said valve assembly further comprises a supplementary check valve, said supplementary check valve cooperatively disposed and structured with said first check valve to establish unidirectional fluid flow of the injected fluid out of said minor chamber, along said first path of fluid flow, towards the abdominal cavity.

12. The assembly as recited in claim 1 further comprising said major chamber operatively positioned within the user's body in accessible relation to an exterior of the user; said major chamber manually disposable between said expanded orientation and said collapsed orientation, via a force applied externally of the user to said major chamber.

13. The assembly as recited in claim 12 wherein said minor chamber is positioned within the user's body in accessible relation to an exterior of the user; said minor chamber disposed and structured to receive the injected fluid therein from an injection instrument operative exteriorly of the user's body.

14. The assembly as recited in claim 1 wherein said conduit assembly comprises a first conduit and a second conduit respectively defining said first path of fluid flow and said second path of fluid flow.

15. The assembly as recited in claim 14 wherein said major chamber and said minor chamber are concurrently connected to said first conduit, via a common connector.

16. The assembly as recited in claim 1 wherein said minor chamber includes a penetration resistant shield disposed and structured to restrict passage of the injection instrument, from an interior of said minor chamber therethrough to an exterior thereof.

17. An assembly implanted in a body of a user operative to remove fluid from the user's abdominal cavity, said assembly comprising:
a major chamber and a minor chamber; said major chamber disposable between a collapsed orientation and an expanded orientation; said minor chamber disposed and structured to receive an injected fluid therein, via an injection instrument,
a conduit assembly comprising a first conduit and a second conduit respectively defining a first path of fluid flow and a second path of fluid flow,
said first conduit interconnecting said major chamber in fluid communication with the abdominal cavity and said second conduit interconnecting said major chamber in fluid communication with a user's bladder,
said major chamber and said minor chamber concurrently connected to said first conduit, via a common connector,
a valve assembly connected to said conduit assembly in flow regulating relation to fluid passing from the abdominal cavity to the bladder, via said major chamber; said valve assembly disposed and structured to define independent fluid communication of said major chamber with the abdominal cavity, via said first path of fluid flow,
said major chamber disposable from said expanded orientation into said collapsed orientation concurrent to fluid flow from said major chamber into the bladder; said major chamber retained in said collapsed orientation concurrent to said first conduit disposed out of direct fluid communication with excess fluid within the abdominal cavity, and
said major chamber disposable from a retained collapsed orientation into said expanded orientation, drawing fluid from the abdominal cavity.

18. The assembly as recited in claim 17 wherein said major chamber is disposable from the retained collapsed orientation into said expanded orientation, upon the injected fluid from said minor chamber disposed in the abdominal cavity.

19. The assembly as recited in claim 17 wherein said major chamber is disposable from a retained collapsed orientation into said expanded orientation, concurrent to being filled, at least in part, with the injected fluid.

20. The assembly as recited in claim 17 wherein said major chamber is disposed into said expanded orientation from a retained collapsed orientation, concurrent to being filled with the injected fluid and peritoneum fluid.
